SEQUENCE LISTING (25 sequences)

SEQ ID NO: 1 - protein, 15 aa, Artificial
  Antibody Light Chain CDR
RASKSVSTSGFSYLH

SEQ ID NO: 2 - protein, 7 aa, Artificial
  Antibody Light Chain CDR
LASNLES

SEQ ID NO: 3 - protein, 9 aa, Artificial
  Antibody Light Chain CDR
QHSWELPLT

SEQ ID NO: 4 - protein, 5 aa, Artificial
  Antibody Heavy Chain CDR
SYYLY

SEQ ID NO: 5 - protein, 17 aa, Artificial
  Antibody Heavy Chain CDR
GVNPSNGGTNFSEKFKS

SEQ ID NO: 6 - protein, 11 aa, Artificial
  Antibody Heavy Chain CDR
RDSNYDGGFDY

SEQ ID NO: 7 - protein, 15 aa, Artificial
  Antibody Light Chain CDR
RASKGVSTSGYSYLH

SEQ ID NO: 8 - protein, 7 aa, Artificial
  Antibody Light Chain CDR
LASYLES

SEQ ID NO: 9 - protein, 9 aa, Artificial
  Antibody Light Chain CDR
QHSRDLPLT

SEQ ID NO: 10 - protein, 5 aa, Artificial
  Antibody Heavy Chain CDR
NYYMY

SEQ ID NO: 11 - protein, 17 aa, Artificial
  Antibody Heavy Chain CDR
GINPSNGGTNFNEKFKN

SEQ ID NO: 12 - protein, 11 aa, Artificial
  Antibody Heavy Chain CDR
RDYRFDMGFDY

SEQ ID NO: 13 - protein, 120 aa, Artificial [sequence omitted]

SEQ ID NO: 14 - protein, 447 aa, Artificial
  Humanized Antibody Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 111 aa, Artificial [sequence omitted]

SEQ ID NO: 16 - protein, 111 aa, Artificial
  Humanized Antibody Light Chain Variable Region
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 17 - protein, 111 aa, Artificial
  Humanized Antibody Light Chain Variable Regon
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 18 - protein, 218 aa, Artificial
  Humanzed Antiboy LIght Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 218 aa, Artificial
  Humanized Antibody Light Chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 218 aa, Artificial
  Humanized Antibody Light Chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, Artificial; identical to SEQ ID NO: 14
  Humanized Antibody Heavy Chain

SEQ ID NO: 22 - protein, 218 aa, Artificial; identical to SEQ ID NO: 18
  Humanized Antibody Light Chain

SEQ ID NO: 23 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

(19) United States
(12) Patent Application Publication
(10) Pub. No.: US 2016/0303231 A1
(43) Pub. Date: Oct. 20, 2016

(54) TREATING CANCER WITH A COMBINATION OF A PD-1 ANTAGONIST AND A VEGFR INHIBITOR

(71) Applicants: Robert IANNONE, Narberth, PA (US); RodoIfo F. PERINI, North Wales, PA (US); Joseph H. PHILLIPS, Palo Alto, CA (US); Sriram VENKATARAMAN, Palo Alto, CA (US); GLAXOSMITHKLINE LLC, Wilmington, DE (US); MERCK SHARP & DOHME CORP., Rahway, NJ (US)

(72) Inventors: Robert Iannone, Narberth, PA (US); Rodolfo F. Perini, North Wales, PA (US); Joseph H. Phillips, Palo Alto, CA (US); Sriram Venkataraman, Palo Alto, CA (US); Elaine Marie Paul, Research Triangle Park, NC (US)

(21) Appl. No.: 15/101,409

(22) PCT Filed: Dec. 3, 2014
(86) PCT No.: PCT/US14/68285

§ 371 Date: Jun. 2, 2016

Related U.S. Application Data
(60) Provisional application No. 61/914,462, filed on Dec. 11, 2013.

(51) Int. Cl. A61K 39/395; A61K 9/20; A61K 47/22; A61K 31/635; A61K 47/26

(52) U.S. Cl. CPC A61K 39/39558; A61K 31/635; A61K 47/26; A61K 47/22; A61K 9/20; A61K 2039/545

(57) ABSTRACT

The present disclosure describes combination therapies comprising an antagonist of Programmed Death 1 receptor (PD-1) and VEGFR inhibitor, and the use of the combination therapies for the treatment of cancer, and in particular for treating cancers that express PD-L1.

### FIELD OF THE INVENTION

The present invention relates to combination therapies useful for the treatment of cancer, to antagonists of a Programmed Death 1 protein (PD-1) and inhibitors of VEGFR kinase.

### BACKGROUND OF THE INVENTION

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (1).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (2-13). Similarly, PD-1 expression on tumor infiltrating lymphocytes was found to mark dysfunctional T cells in breast cancer and melanoma (14-15) and to correlate with poor prognosis in renal cancer (16). Thus, it has been proposed that PD-L1 expressing tumor cells interact with PD-1 expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 are in clinical development for treating cancer. It has been proposed that the efficacy of such antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents.

Apart from immune regulation, one of the most commonly studied pathways of cancerous deregulation of the cell division process, which involves kinase regulation of apoptosis, is cellular signaling from growth factor receptors at the cell surface to the nucleus (Crews and Erikson, Cell, 74:215-17, 1993).

The process of angiogenesis is the development of new blood vessels from the pre-existing vasculature. Angiogenesis is defined herein as involving: (i) activation of endothelial cells; (ii) increased vascular permeability; (iii) subsequent dissolution of the basement membrane and extravasation of plasma components leading to formation of a provisional fibrin gel extracellular matrix; (iv) proliferation and mobilization of endothelial cells; (v) reorganization of mobilized endothelial cells to form functional capillaries; (vi) capillary loop formation; and (vi) deposition of basement membrane and recruitment of perivascular cells to newly formed vessels. Normal angiogenesis is active during tissue growth from embryonic development through maturity and then enters a period of relative quiescence during adulthood. Normal angiogenesis is also activated during wound healing, and at certain stages of the female reproductive cycle. Inappropriate or pathological angiogenesis has been associated with several disease states including various retinopathies, ischemic disease, atherosclerosis, chronic inflammatory disorders, and cancer. The role of angiogenesis in disease states is discussed, for instance, in Fan et al., Trends in Pharmacol Sci. 16:54-66; Shawver et al., DDT Vol. 2, No. 2 Feb. 1997; Folkmann, 1995, Nature Medicine 1:27-31.

In cancer the growth of solid tumors has been shown to be dependent on angiogenesis. The progression of leukemias as well as the accumulation of fluid associated with malignant ascites and pleural effusions also involve pro-angiogenic factors. (See Folkmann, J., J. Nat'l. Cancer Inst, 1990, 82, 4-6).

Central to the process of angiogenesis are vascular endothelial growth factor (VEGF) and its receptors, termed vascular endothelial growth factor receptor(s) (VEGFRs). The roles VEGF and VEGFRs play in the vascularization of solid tumors, progression of hematopoietic cancers and modulation of vascular permeability have drawn great interest in the scientific community. VEGF is a polypeptide, which has been linked to inappropriate or pathological angiogenesis (Pinedo, H. M. et al. The Oncologist, Vol. 5, No. 90001, 1-2, Apr. 2000). VEGFR(s) are protein tyrosine kinases (PTKs) that catalyze the phosphorylation of specific tyrosine residues in proteins that are involved in the regulation of cell growth, differentiation, and survival. (A. F. Wilks, Progress in Growth Factor Research, 1990, 2, 97-111; S. A. Courtneidge, Dev. Supp. 1, 1993, 57-64; J. A. Cooper, Semin. Cell Biol., 1994, 5(6),377-387; R. F. Paulson, Semin. Immunol. 1995, 7(4), 267-277; A. C. Chan, Curr. Opin. Immunol. 1996, 8(3), 394-401). Three PTK receptors for VEGF have been identified: VEGFRI (FIt-I); VEGFR2 (Flk-I and KDR) and VEGFR3 (Flt-4). These receptors are involved in angiogenesis and participate in signal transduction. (Mustonen, T. et al. J. Cell. Biol. 1995: 129:895-898; Ferrara and Davis-Smyth, Endocrine Reviews, 18(1):4-25, 1997; McMahon, G., The Oncologist, Vol. 5, No 90001, 3-10, Apr. 2000).

Of particular interest is VEGFR2, which is a transmembrane receptor PTK expressed primarily in endothelial cells. Activation of VEGFR-2 by VEGF is a critical step in the signal transduction pathway that initiates tumor angiogenesis. VEGF expression may be constitutive to tumor cells and can also be upregulated in response to certain stimuli. One such stimulus is hypoxia, where VEGF expression is upregulated in both tumor and associated host tissues. The VEGF ligand activates VEGFR2 by binding to its extracellular VEGF binding site. This leads to receptor dimerization of VEGFRs and autophosphorylation of tyrosine residues at the intracellular kinase domain of VEGFR2. The kinase domain operates to transfer a phosphate from ATP to the tyrosine residues, thus providing binding sites for signaling proteins downstream of VEGFR-2 leading ultimately to angiogenesis. (Ferrara and Davis-Smyth, Endocrine Reviews, 18(1):4-25, 1997; McMahon, G. The Oncologist, Vol. 5, No. 90001, 3-10, Apr. 2000.)

Consequently, antagonism of the VEGFR2 kinase domain would block phosphorylation of tyrosine residues and serve to disrupt initiation of angiogenesis. Specifically, inhibition at the ATP binding site of the VEGFR2 kinase domain would prevent binding of ATP and prevent phosphorylation of tyrosine residues. Such disruption of the proangiogenesis signal transduction pathway associated with VEGFR2 should therefore inhibit tumor angiogenesis and thereby provide a potent treatment for cancer or other disorders associated with inappropriate angiogenesis.

Votrient (pazopanib hydrochloride) is a multi-tyrosine kinase inhibitor of vascular endothelial growth factor receptor (VEGFR)-1, VEGFR-2, VEGFR-3, platelet-derived growth factor receptor (PDGFR)-α and -β, fibroblast growth factor receptor (FGFR)-1 and -3, cytokine receptor (Kit), interleukin-2 receptor inducible T-cell kinase (Itk), leukocyte-specific protein tyrosine kinase (Lck), and transmembrane glycoprotein receptor tyrosine kinase (c-Fms) and is approved in the US for the treatment of patients with advanced renal cell carcinoma. The chemical name of pazopanib hydrochloride is 5-[[4-[(2,3-dimethyl-2H-indazol-6-yl)methylamino]-2-pyrimidinyl]amino]-2-methylbenzenesulfonamide monohydrochloride.

### SUMMARY OF THE INVENTION

In one embodiment, the invention provides a method for treating a cancer in an individual comprising administering to the individual a combination therapy which comprises a PD-1 antagonist and a VEGFR inhibitor.

In another embodiment, the invention provides a medicament comprising a PD-1 antagonist for use in combination with a VEGFR inhibitor for treating a cancer.

In yet another embodiment, the invention provides a medicament comprising a VEGFR inhibitor for use in combination with a PD-1 antagonist for treating a cancer.

Other embodiments provide use of a PD-1 antagonist in the manufacture of medicament for treating a cancer in an individual when administered in combination with a VEGFR inhibitor and use of a VEGFR inhibitor in the manufacture of a medicament for treating a cancer in an individual when administered in combination with a PD-1 antagonist.

In a still further embodiment, the invention provides use of a PD-1 antagonist and a VEGFR inhibitor in the manufacture of medicaments for treating a cancer in an individual. In some preferred embodiments, the medicaments comprise a kit, and the kit also comprises a package insert comprising instructions for using the PD-1 antagonist in combination with a VEGFR inhibitor to treat a cancer in an individual.

In all of the above treatment method, medicaments and uses, the PD-1 antagonist inhibits the binding of PD-L1 to PD-1, and preferably also inhibits the binding of PD-L2 to PD-1. In some preferred embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one particularly preferred embodiment, the PD-1 antagonist is an anti-PD-1 antibody which comprises a heavy chain and a light chain, and wherein the heavy and light chains comprise the amino acid sequences shown in FIG. 6 (SEQ ID NO:21 and SEQ ID NO:22).

In all of the above embodiments of the treatment method, medicaments and uses, the VEGFR inhibitor is the compound of Formula I

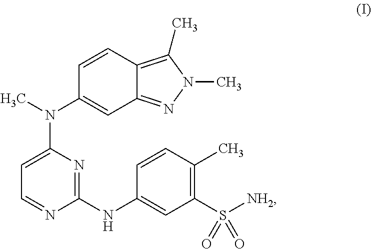 [SMILES: Cc1ccc(Nc2nccc(N(C)c3ccc4c(C)n(C)nc4c3)n2)cc1S(N)(=O)=O]

or a pharmaceutically acceptable salt thereof.

In some embodiments of the above treatment method, medicaments and uses of the invention, the individual is a human and the cancer is a solid tumor and in some preferred embodiments, the solid tumor is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell cancer, small-cell lung cancer (SCLC) or triple negative breast cancer. In some preferred embodiments, the cancer is renal cell carcinoma.

In other embodiments of the above treatment method, medicaments and uses of the invention, the individual is a human and the cancer is a Heme malignancy and in some preferred embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, pimary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

Also, in preferred embodiments of any of the above treatment method, medicaments and uses, the cancer expresses one or both of PD-L1 and PD-L2. In particularly preferred embodiments, PD-L1 expression is elevated in the cancer.

In one particularly preferred embodiment of the above treatment method, medicaments and uses, the individual is a human and the cancer is renal cell carcinoma that expresses human PD-L1.

### BRIEF DESCRIPTION OF THE DRAWINGS

FIG. 1 shows amino acid sequences of the light chain and heavy chain CDRs for an exemplary anti-PD-1 monoclonal antibody useful in the present invention (SEQ ID NOs:1-6).

FIG. 2 shows amino acid sequences of the light chain and heavy chain CDRs for another exemplary anti-PD-1 monoclonal antibody useful in the present invention (SEQ ID NOs:7-12).

FIG. 3 shows amino acid sequences of the heavy chain variable region and full length heavy chain for an exemplary anti-PD-1 monoclonal antibody useful in the present invention (SEQ ID NO:13 and SEQ ID NO:14).

FIG. 4 shows amino acid sequences of alternative light chain variable regions for an exemplary anti-PD-1 monoclonal antibody useful in the present invention (SEQ ID NOs:15-17).

FIG. 5 shows amino acid sequences of alternative light chains for an exemplary anti-PD-1 monoclonal antibody useful in the present invention (SEQ ID NOs:18-20).

FIG. 6 shows amino acid sequences of the heavy and light chains for MK-3475 (SEQ ID NOs. 21 and 22, respectively).

FIG. 7 shows amino acid sequences of the heavy and light chains for nivolumab (SEQ ID NOs. 23 and 24, respectively).

FIG. 8 shows the dosing scheme for the mouse studies of Example 1.

FIG. 9 shows results data for the mouse studies of Example 1 with once a day pazopanib dosing.

FIG. 10 shows the results data for the mouse studies of Example 1 with twice a day pazopanib dosing.

### DETAILED DESCRIPTION

### Abbreviations

Throughout the detailed description and examples of the invention the following abbreviations will be used:

CDR Complementarity determining region

CHO Chinese hamster ovary

DTR Dose limiting toxicity

FFPE Formalin-fixed, paraffin-embedded

FR Framework region

IgG Immunoglobulin G

IHC Immunohistochemistry or immunohistochemical

MTR Maximum Tolerated Regimen

Q2W One dose every two weeks

Q3W One dose every three weeks

VH Immunoglobulin heavy chain variable region

VK Immunoglobulin kappa light chain variable region

### I. DEFINITIONS

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). As used herein, the term “framework” or “FR” residues refers to those variable domain residues other than the hypervariable region residues defined herein as CDR residues.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Particularly preferred cancers that may be treated in accordance with the present invention include those characterized by elevated expression of one or both of PD-L1 and PD-L2 in tested tissue samples.

“Biotherapeutic agent” means a biological molecule, such as an antibody or fusion protein, that blocks ligand/receptor signaling in any biological pathway that supports tumor maintenance and/or growth or suppresses the anti-tumor immune response.

“CDR” or “CDRs” as used herein means complementarity determining region(s) in a immunoglobulin variable region, defined using the Kabat numbering system, unless otherwise indicated

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth. Chemotherapeutic agents useful in the treatment methods of the present invention include cytostatic and/or cytotoxic agents.

“Clothia” as used herein means an antibody numbering system described in Al-Lazikani et al., JMB 273:927-948 (1997).

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antagonist that consists essentially of a recited amino acid sequence may also include one or more amino acids, including substitutions of one or more amino acid residues, which do not materially affect the properties of the binding compound.

“Diagnostic anti-PD-L monoclonal antibody” means a mAb which specifically binds to the mature form of the designated PD-L (PD-L1 or PDL2) that is expressed on the surface of certain mammalian cells. A mature PD-L lacks the presecretory leader sequence, also referred to as leader peptide The terms “PD-L” and “mature PD-L” are used interchangeably herein, and shall be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 25)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET.

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in the copending U.S. provisional patent application 61/745,386, filed December 2012. Another anti-human PD-L1 mAb that has been reported to be useful for IHC detection of PD-L1 expression in FFPE tissue sections (Chen, B. J. et al., Clin Cancer Res 19: 3462-3473 (2013)) is a rabbit anti-human PD-L1 mAb publicly available from Sino Biological, Inc. (Beijing, P.R. China; Catalog number 10084-R015).

DLT means a drug-related adverse event observed in a patient starting in the first 8 weeks of treatment with a PD-1 antagonist (e.g., MK-3475) in combination with a VEGFR inhibitor (e.g., pazopanib).

MTR means the highest dose of a PD-1 antagonist (e.g., MK-3475) in combination with the highest does of a VEGFR inhibitor (e.g., pazopanib) at which no more than 1 of 6 subjects experiences a DLT after a minimum of 8 weeks of treatment with the combination.

“VEGFR inhibitor” means the compound of Formula I, and pharmaceutically acceptable salts of the compound of Formula I. The compound of formula (I) is disclosed and claimed, along with pharmaceutically acceptable salts thereof, as being useful as an inhibitor of VEGFR activity, particularly in treatment of cancer, in International Application No. PCT/US01/49367, having an International filing date of Dec. 19, 2001, International Publication Number WO02/059110 and an International Publication date of Aug. 1, 2002, the entire disclosure of which is hereby incorporated by reference. The compound can be prepared as described in International Application No. PCT/US01/49367, or any other synthetic route that will be readily apparent to the skilled artisan.

Suitably, the compound of formula (I) is in the form of a monohydrochloride salt. This salt form can be prepared by one of skill in the art from the description in International Application No. PCT/US01/49367, having an International filing date of Dec. 19, 2001.

The compound of formula (I) is known by the generic name pazopanib and the trade name Votrient®.

The salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention. Salts may be readily prepared by a person skilled in the art.

Pharmaceutical compositions may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage compositions are those containing a daily dose or sub-dose, or an appropriate fraction thereof, of an active ingredient. Furthermore, such pharmaceutical compositions may be prepared by any of the methods well known in the pharmacy art.

Prodrugs of the compound of Formula I are also contemplated for use in the methods, medicaments and uses of the present invention. The term “prodrug”, as employed herein, denotes a compound that is a drug precursor which, upon administration to a subject, undergoes chemical conversion by metabolic or chemical processes to yield a compound of Formula I or a salt thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Framework region” or “FR” as used herein means the immunoglobulin variable regions excluding the CDR regions.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.″ M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Patient” or “subject” refers to any single subject for which therapy is desired or that is participating in a clinical trial, epidemiological study or used as a control, including humans and mammalian veterinary patients such as cattle, horses, dogs, and cats.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the any of the treatment method, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

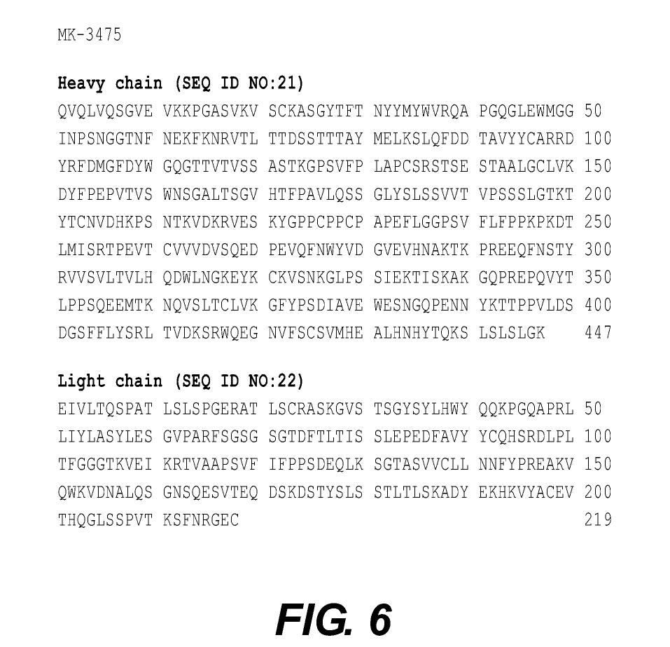

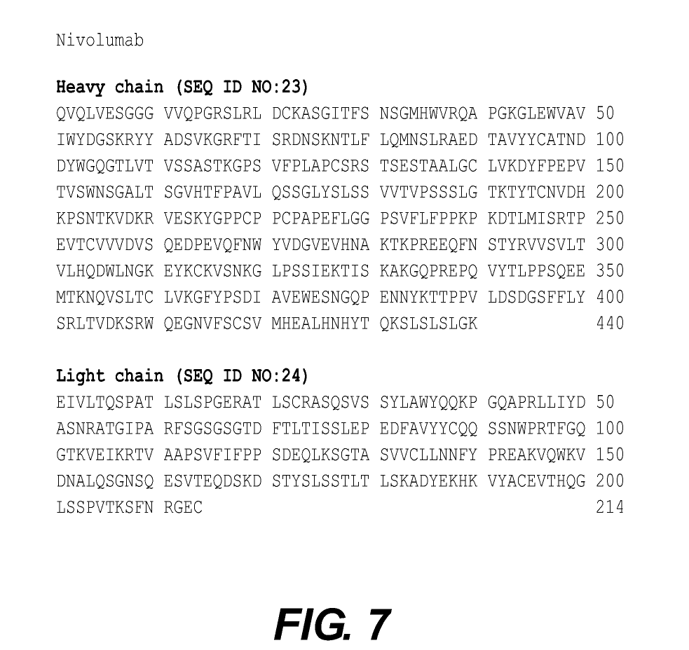

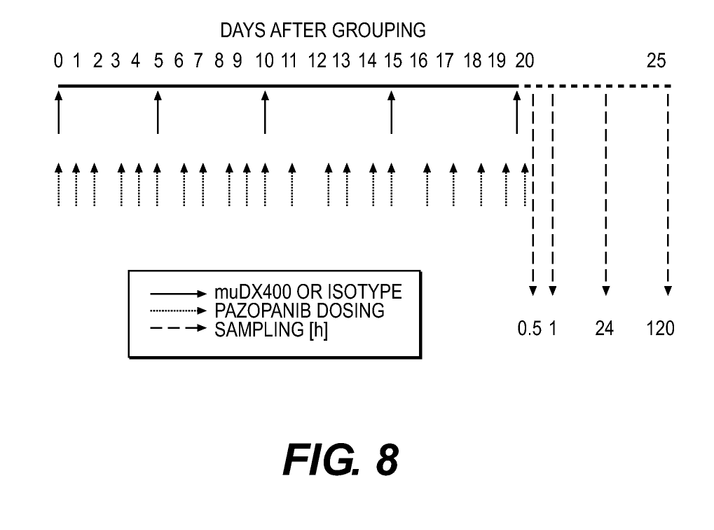

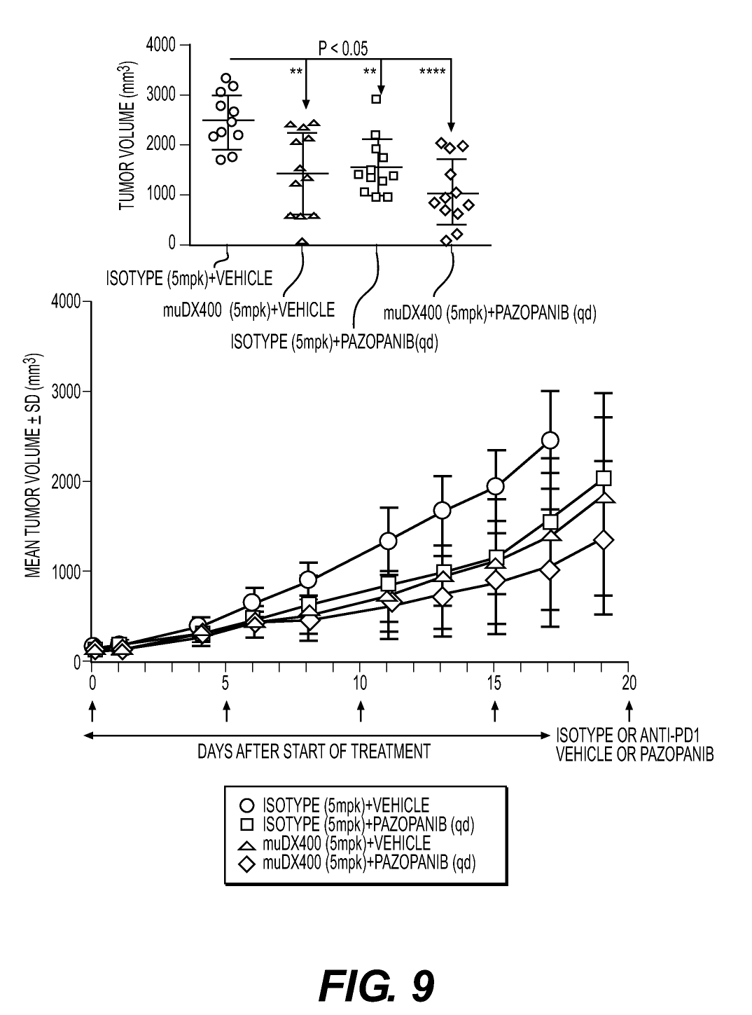

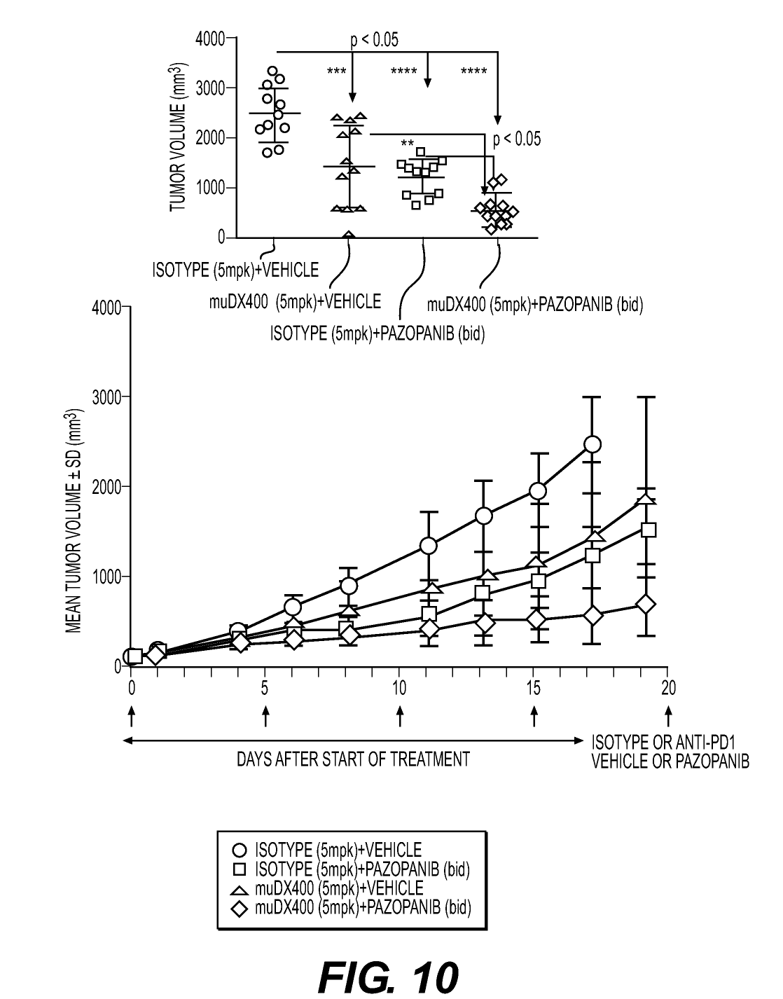

Examples of mAbs that bind to human PD-1, and useful in the treatment method, medicaments and uses of the present invention, are described in U.S. Pat. No. 7,521,051, U.S. Pat. No. 8,008,449, and U.S. Pat. No. 8,354,509. Specific anti-human PD-1 mAbs useful as the PD-1 antagonist in the treatment method, medicaments and uses of the present invention include: MK-3475, a humanized IgG4 mAb with the structure described in WHO Drug Information, Vol. 27, No. 2, pages 161-162 (2013) and which comprises the heavy and light chain amino acid sequences shown in FIG. 6, nivolumab (BMS-936558), a human IgG4 mAb with the structure described in WHO Drug Information, Vol. 27, No. 1, pages 68-69 (2013) and which comprises the heavy and light chain amino acid sequences shown in FIG. 7; the humanized antibodies h409A11, h409A16 and h409A17, which are described in WO2008/156712, and AMP-514.

Examples of mAbs that bind to human PD-L1, and useful in the treatment method, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in the any of the treatment method, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

In some preferred embodiments of the treatment method, medicaments and uses of the present invention, the PD-1 antagonist is a monoclonal antibody, or antigen binding fragment thereof, which comprises: (a) light chain CDRs SEQ ID NOs: 1, 2 and 3 and heavy chain CDRs SEQ ID NOs: 4, 5 and 6; or (b) light chain CDRs SEQ ID NOs: 7, 8 and 9 and heavy chain CDRs SEQ ID NOs: 10, 11 and 12.

In other preferred embodiments of the treatment method, medicaments and uses of the present invention, the PD-1 antagonist is a monoclonal antibody, or antigen binding fragment thereof, which specifically binds to human PD-1 and comprises (a) a heavy chain variable region comprising SEQ ID NO:13 or a variant thereof, and (b) a light chain variable region comprising an amino acid sequence selected from the group consisting of SEQ ID NO:15 or a variant thereof; SEQ ID NO:16 or a variant thereof; and SEQ ID NO: 17 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment method, medicaments and uses of the present invention, the PD-1 antagonist is a monoclonal antibody which specifically binds to human PD-1 and comprises (a) a heavy chain comprising SEQ ID NO: 14 and (b) a light chain comprising SEQ ID NO:18, SEQ ID NO:19 or SEQ ID NO:20.

In yet another preferred embodiment of the treatment method, medicaments and uses of the present invention, the PD-1 antagonist is a monoclonal antibody which specifically binds to human PD-1 and comprises (a) a heavy chain comprising SEQ ID NO: 14 and (b) a light chain comprising SEQ ID NO:18.

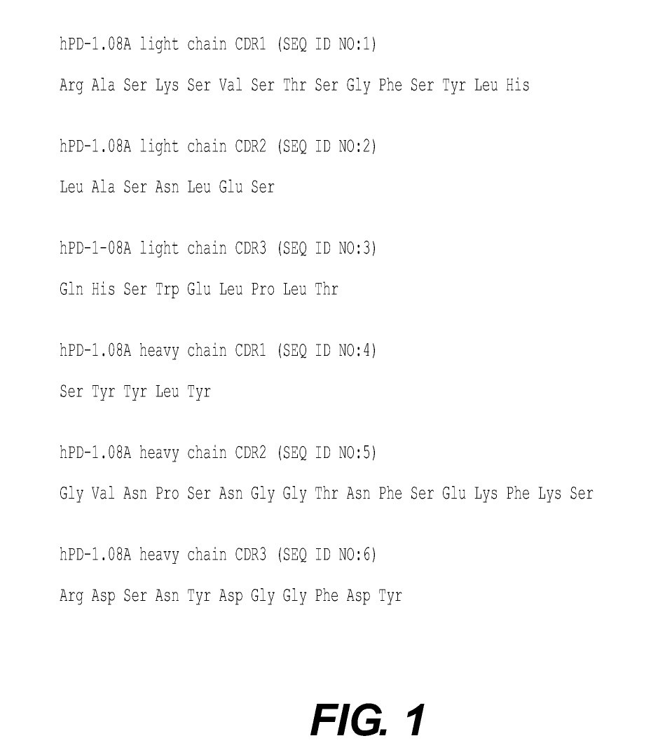

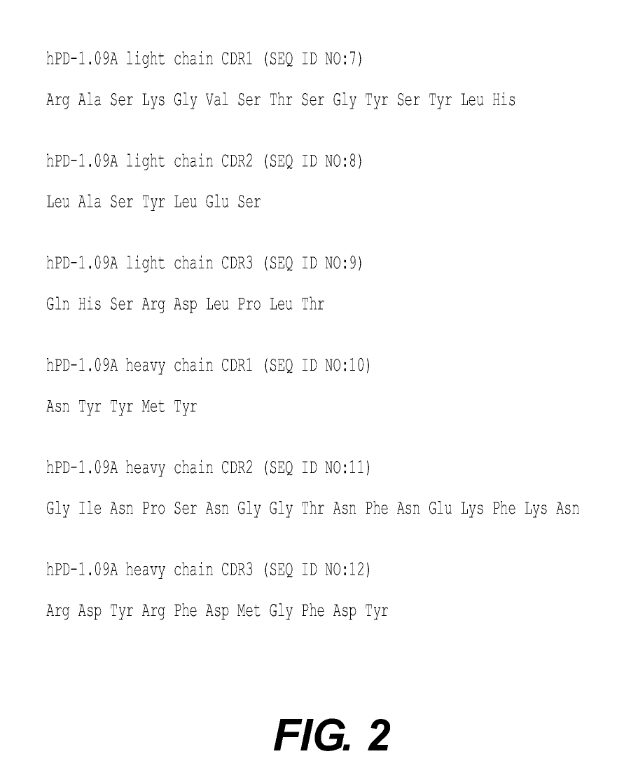

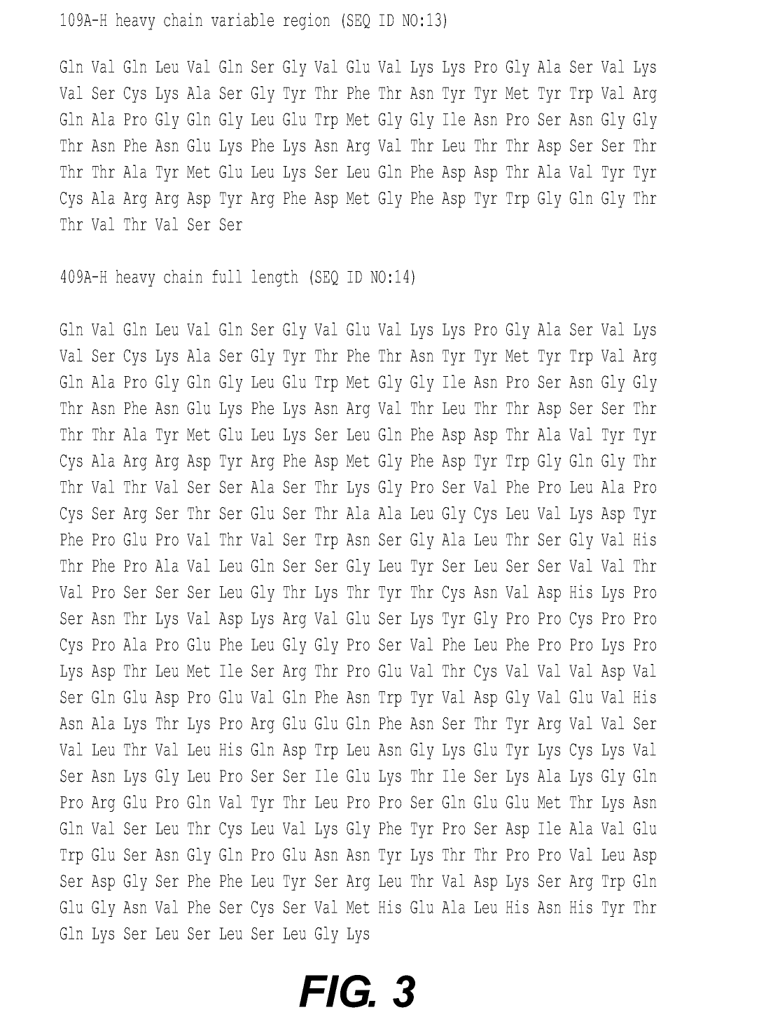

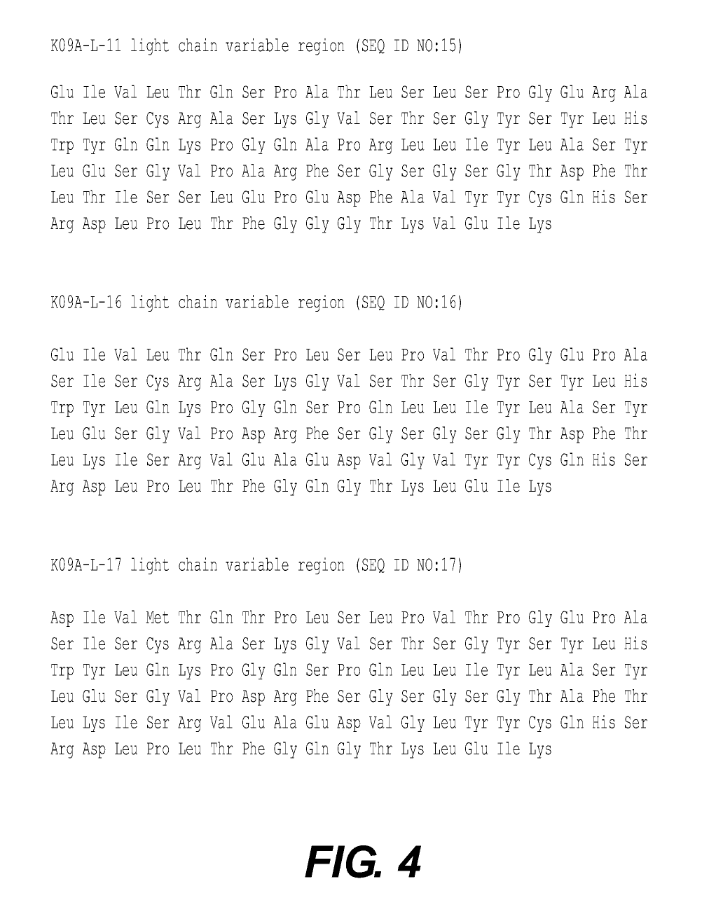

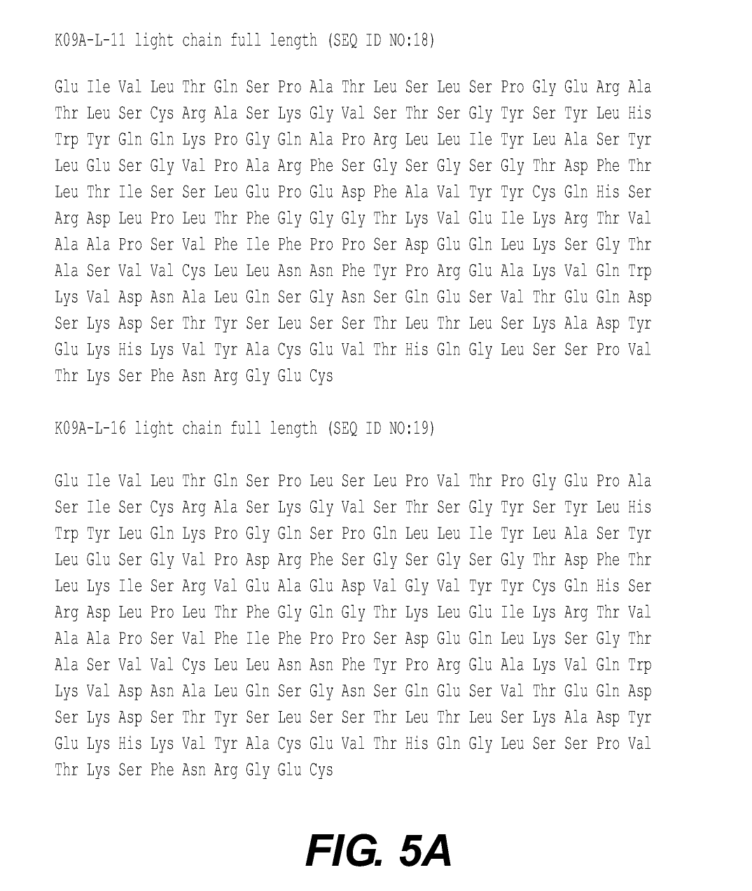

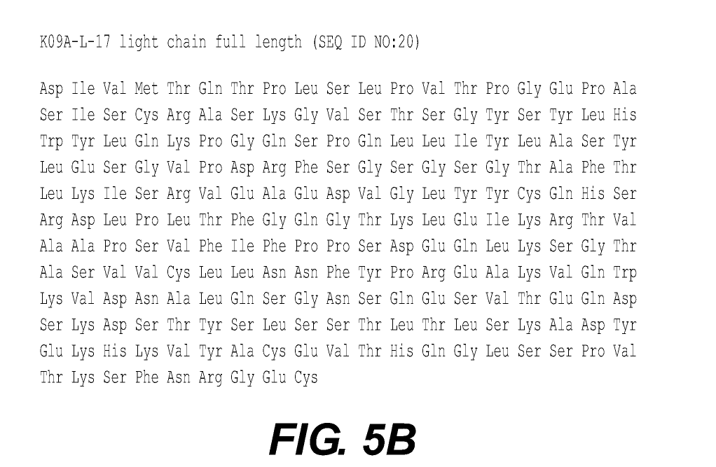

Table 3 below provides a list of the amino acid sequences of exemplary anti-PD-1 mAbs for use in the treatment method, medicaments and uses of the present invention, and the sequences are shown in FIGS. 1-5.

TABLE 3
Exemplary anti-human PD-1 antibodies
A. Comprises light and heavy chain CDRs of hPD-1.08A in
WO2008/156712

| | |
|---|---|
| CDRL1 | SEQ ID NO: 1 |
| CDRL2 | SEQ ID NO: 2 |
| CDRL3 | SEQ ID NO: 3 |
| CDRH1 | SEQ ID NO: 4 |
| CDRH2 | SEQ ID NO: 5 |
| CDRH3 | SEQ ID NO: 6 |

B. Comprises light and heavy chain CDRs of hPD-1.09A in
WO2008/156712

| | |
|---|---|
| CDRL1 | SEQ ID NO: 7 |
| CDRL2 | SEQ ID NO: 8 |
| CDRL3 | SEQ ID NO: 9 |
| CDRH1 | SEQ ID NO: 10 |
| CDRH2 | SEQ ID NO: 11 |
| CDRH3 | SEQ ID NO: 12 |

C. Comprises the mature h109A heavy chain variable region and one of
the mature K09A light chain variable regions in WO2008/156712

| | |
|---|---|
| Heavy chain | SEQ ID NO: 13 |
| VR | |
| Light chain | SEQ ID NO: 15 or SEQ ID NO: 16 or SEQ ID NO: 17 |
| VR | |

D. Comprises the mature 409 heavy chain and one of the mature K09A
light chains in WO2008/156712

| | |
|---|---|
| Heavy chain | SEQ ID NO: 14 |
| Light chain | SEQ ID NO: 18 or SEQ ID NO: 19 or SEQ ID NO: 20 |

“PD-L1” or “PD-L2” expression as used herein means any detectable level of expression of the designated PD-L protein on the cell surface or of the designated PD-L mRNA within a cell or tissue. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR and realtime quantitative RT-PCR.

Several approaches have been described for quantifying PD-L1 protein expression in IHC assays of tumor tissue sections. See, e.g., Thompson, R. H., et al., PNAS 101 (49); 17174-17179 (2004); Thompson, R. H. et al., Cancer Res. 66:3381-3385 (2006); Gadiot, J., et al., Cancer 117:2192-2201 (2011); Taube, J. M. et al., Sci Transl Med 4, 127ra37 (2012); and Toplian, S. L. et al., New Eng. J Med. 366 (26): 2443-2454 (2012).

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is ≧5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the MS is ≧5.

The level of PD-L mRNA expression may be compared to the mRNA expression levels of one or more reference genes that are frequently used in quantitative RT-PCR, such as ubiquitin C.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In some preferred embodiments, PD-L1 expression in a tumor sample is determined to be elevated if PD-L1 protein (and/or PD-L1 mRNA) in the sample is at least 10%, 20%, or 30% greater than in the control.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” a cancer as used herein means to administer a combination therapy of a PD-1 antagonist and a VEGFR inhibitor to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to NCI standards, a T/C≦42% is the minimum level of anti-tumor activity. A T/C<10% is considered a high anti-tumor activity level, with T/C (%)=Median tumor volume of the treated/Median tumor volume of the control×100. In some embodiments, the treatment achieved by a therapeutically effective amount is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. The dosage regimen of a combination therapy described herein that is effective to treat a cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Variable regions” or “V region” as used herein means the segment of IgG chains which is variable in sequence between different antibodies. It extends to Kabat residue 109 in the light chain and 113 in the heavy chain.

### II. METHODS, USES AND MEDICAMENTS

In one aspect of the invention, the invention provides a method for treating a cancer in an individual comprising administering to the individual a combination therapy which comprises a PD-1 antagonist and a VEGFR inhibitor.

The combination therapy may also comprise one or more additional therapeutic agents. The additional therapeutic agent may be, e.g., a chemotherapeutic other than a VEGFR inhibitor, a biotherapeutic agent (including but not limited to antibodies to VEGF, EGFR, Her2/neu, VEGF receptors, other growth factor receptors, CD20, CD40, CD-40L, CTLA-4, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, the VEGFR inhibitor is administered before administration of the PD-1 antagonist, while in other embodiments, the VEGFR inhibitor is administered after administration of the PD-1 antagonist.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy of the invention can be administered orally or parenterally, including the intravenous, intramuscular, intraperitoneal, subcutaneous, rectal, topical, and transdermal routes of administration.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naïve. In other embodiments, the combination therapy is administered to a patient who failed to achieve a sustained response after prior therapy with a biotherapeutic or chemotherapeutic agent, i.e., is treatment-experienced.

A combination therapy of the invention is typically used to treat a tumor that is large enough to be found by palpation or by imaging techniques well known in the art, such as MRI, ultrasound, or CAT scan. In some preferred embodiments, a combination therapy of the invention is used to treat an advanced stage tumor having dimensions of at least about 200 mm³, 300 mm³, 400 mm³, 500 mm³, 750 mm³, or up to 1000 mm³.

A combination therapy of the invention is preferably administered to a human patient who has a cancer that tests positive for PD-L1 expression. In some preferred embodiments, PD-L1 expression is detected using a diagnostic anti-human PD-L1 antibody, or antigen binding fragment thereof, in an IHC assay on an FFPE or frozen tissue section of a tumor sample removed from the patient. Typically, the patient's physician would order a diagnostic test to determine PD-L1 expression in a tumor tissue sample removed from the patient prior to initiation of treatment with the PD-1 antagonist and VEGFR inhibitor, but it is envisioned that the physician could order the first or subsequent diagnostic tests at any time after initiation of treatment, such as for example after completion of a treatment cycle.

In some embodiments, combination therapy with a PD-1 antagonist and a VEGFR inhibitor is indicated for treating a human patient diagnosed with locally advanced (defined as disease not amenable to curative surgery or radiation therapy) or metastatic RCC (equivalent to Stage IV RCC according to American Joint Committee on Cancer [AJCC] staging) that is predominantly clear cell histology. Preferably, the patient has measurable disease, i.e. presents with at least one measurable lesion, and even more preferably, the patient is treatment naïve for one or both of the PD-1 antagonist and the VEGFR inhibitor.

In some embodiments, a human patient treated with a combination of PD-1 antagonist and a VEGFR inhibitor does not have a pre-treatment diagnosis or history of any of the following conditions: an autoimmune disease that requires treatment with systemic steroid or immunosuppressive agents, interstitial lung disease, pneumonitis, Malabsorption syndrome, active peptic ulcer disease, inflammatory bowel disease, ulcerative colitis, any gastrointestinal (GI) condition with increased risk of perforation; history of abdominal fistula, GI perforation, or intra-abdominal abscess.

Selecting a dosage regimen (also referred to herein as an administration regimen) for a combination therapy of the invention depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

In some embodiments that employ an anti-human PD-1 mAb as the PD-1 antagonist in the combination therapy, the dosing regimen will comprise administering the anti-human PD-1 mAb at a dose of 1, 2, 3, 5 or 10 mg/kg at intervals of about 14 days (±2 days) or about 21 days (±2 days) or about 30 days (±2 days) throughout the course of treatment.

In other embodiments that employ an anti-human PD-1 mAb as the PD-1 antagonist in the combination therapy, the dosing regimen will comprise administering the anti-human PD-1 mAb at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In certain embodiments, a subject will be administered an intravenous (IV) infusion of a medicament comprising any of the PD-1 antagonists described herein.

In one preferred embodiment of the invention, the PD-1 antagonist in the combination therapy is nivolumab, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W.

In another preferred embodiment of the invention, the PD-1 antagonist in the combination therapy is MK-3475, which is administered in a liquid medicament at a dose selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W. In some particularly preferred embodiments, MK-3475 is administered as a liquid medicament which comprises 25 mg/ml MK-3475, 7% (w/v) sucrose, 0.02% (w/v) polysorbate 80 in 10 mM histidine buffer pH 5.5, and the selected dose of the medicament is administered by IV infusion over a time period of 30 minutes. The optimal dose for MK-3475 in combination with pazopanib may be identified by dose escalation. According to an embodiment, pazopanib is taken once a day at about the same time each day while MK-3475 is administered every 2 weeks or every 3 weeks. On the day of MK-3475 administration, pazopanib is advantageously taken before MK-3475, but may occur in reverse order. According to the preceding embodiment, pazopanib is advantageously orally administered as a 200 mg tablet to be administered in the dose range of 400 to 800 mg. According to the embodiment, MK 3475 is advantageously an intravenously administered 100 mg/4 mL solution available in the potential dose range of 1 to 10 mg/kg.

In some embodiments, the compound of formula (I) is prepared as the hydrochloride salt in tablet form with unit dosage of 200 mg by free base. In some embodiments, the compound of formula (I) is dosed to a human 800 mg once daily.

The present invention also provides a medicament which comprises a PD-1 antagonist as described above and a pharmaceutically acceptable excipient. When the PD-1 antagonist is a biotherapeutic agent, e.g., a mAb, the antagonist may be produced in CHO cells using conventional cell culture and recovery/purification technologies.

In some embodiments, a medicament comprising an anti-PD-1 antibody as the PD-1 antagonist may be provided as a liquid formulation or prepared by reconstituting a lyophilized powder with sterile water for injection prior to use. WO 2012/135408 describes the preparation of liquid and lyophilized medicaments comprising MK-3475 that are suitable for use in the present invention. In some preferred embodiments, a medicament comprising MK-3475 is provided in a glass vial which contains about 50 mg of MK-3475.

The anti-PD-1 and VEGFR inhibitor medicaments described herein may be provided as a kit which comprises a first container and a second container and a package insert. The first container contains at least one dose of a medicament comprising an anti-PD-1 antagonist, the second container contains at least one dose of a medicament comprising a VEGFR inhibitor, and the package insert, or label, which comprises instructions for treating a patient for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some preferred embodiments of the kit, the anti-PD-1 antagonist is an anti-PD-1 antibody and the instructions state that the medicaments are intended for use in treating a patient having a cancer that tests positive for PD-L1 expression by an IHC assay.

These and other aspects of the invention, including the exemplary specific embodiments listed below, will be apparent from the teachings contained herein.

### Exemplary Specific Embodiments of the Invention

1. A method for treating a cancer in an individual comprising administering to the individual a combination therapy which comprises a PD-1 antagonist and a VEGFR inhibitor, wherein the VEGFR inhibitor is the compound of Formula I

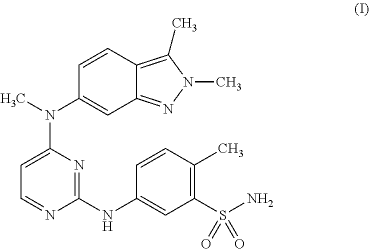 [SMILES: Cc1ccc(Nc2nccc(N(C)c3ccc4c(C)n(C)nc4c3)n2)cc1S(N)(=O)=O]

or a pharmaceutically acceptable salt of the compound of Formula I.
2. A medicament comprising a PD-1 antagonist for use in combination with a VEGFR inhibitor for treating a cancer in an individual.
3. A medicament comprising a VEGFR inhibitor for use in combination with a PD-1 antagonist for treating a cancer in an individual.
4. The medicament of embodiment 3 or 4, which further comprises a pharmaceutically acceptable excipient.
5. Use of a PD-1 antagonist in the manufacture of medicament for treating a cancer in an individual when administered in combination with a VEGFR inhibitor.
6. Use of a VEGFR inhibitor in the manufacture of a medicament for treating a cancer in an individual when administered in combination with a PD-1 antagonist.
7. Use of a PD-1 antagonist and a VEGFR inhibitor in the manufacture of medicaments for treating a cancer in an individual.
8. A kit which comprises a first container, a second container and a package insert, wherein the first container comprises at least one dose of a medicament comprising an anti-PD-1 antagonist, the second container comprises at least one dose of a medicament comprising a VEGFR inhibitor, and the package insert comprises instructions for treating an individual for cancer using the medicaments.
9. The kit of embodiment 8, wherein the instructions state that the medicaments are intended for use in treating an individual having a cancer that tests positive for PD-L1 expression by an immunohistochemical (IHC) assay.
10. The method, medicament, use or kit of any of embodiments 1 to 9, wherein the individual is a human and the PD-1 antagonist is a monoclonal antibody, or an antigen binding fragment thereof, which specifically binds to human PD-L1 and blocks the binding of human PD-L1 to human PD-1.
11. The method, medicament, use or kit of embodiment 9, wherein the PD-1 antagonist is MPDL3280A, BMS-936559, MEDI4736, MSB0010718C or a monoclonal antibody which comprises the heavy chain and light chain variable regions of SEQ ID NO:24 and SEQ ID NO:21, respectively, of WO2013/019906.
12. The method, medicament, use or kit of any of embodiments 1 to 9, wherein the individual is a human, and the PD-1 antagonist is a monoclonal antibody, or an antigen binding fragment thereof, which specifically binds to human PD-1 and blocks the binding of human PD-L1 to human PD-1.
13. The method, medicament, use or kit of embodiment 12, wherein the PD-1 antagonist also blocks binding of human PD-L2 to human PD-1.
14. The method, medicament, use or kit of embodiment 13, wherein the monoclonal antibody, or antigen binding fragment thereof, comprises: (a) light chain CDRs of SEQ ID NOs: 1, 2 and 3 and heavy chain CDRs of SEQ ID NOs: 4, 5 and 6; or (b) light chain CDRs of SEQ ID NOs: 7, 8 and 9 and heavy chain CDRs of SEQ ID NOs: 10, 11 and 12.
15. The method, medicament, use or kit of embodiment 13, wherein the monoclonal antibody, or antigen binding fragment thereof, comprises light chain CDRs of SEQ ID NOs: 7, 8 and 9 and heavy chain CDRs of SEQ ID NOs: 10, 11 and 12.
16. The method, medicament, use or kit of embodiment 13, wherein the PD-1 antagonist is an anti-PD-1 monoclonal antibody which comprises a heavy chain and a light chain, and wherein the heavy chain comprises SEQ ID NO:21 and the light chain comprises SEQ ID NO:22.
17. The method, medicament, use or kit of embodiment 13, wherein the PD-1 antagonist is an anti-PD-1 monoclonal antibody which comprises a heavy chain and a light chain, and wherein the heavy chain comprises SEQ ID NO:23 and the light chain comprises SEQ ID NO:24.
18. The method, medicament, use or kit of any of embodiments 10-17, wherein the cancer is a solid tumor.
19. The method, medicament, use or kit of any of embodiments 10-17, wherein the cancer is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell cancer, small-cell lung cancer (SCLC) or triple negative breast cancer.
20. The method, medicament, use or kit of any of embodiments 10-17, wherein the cancer is renal cell carcinoma.
21. The method, medicament, use or kit of any of embodiments 10-17, wherein the cancer is advanced renal cell carcinoma.
22. The method, medicament, use or kit of any of embodiments 10-17, wherein the cancer is acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).
23. The method, medicament, use or kit of any of embodiments 10-22, the cancer expresses human PD-L1.
24. The method, medicament, use or kit of embodiment 25, wherein the human PD-L1 expression is elevated.
25. The method, medicament, use or kit of embodiment 16, wherein the PD-1 antagonist is MK-3475 or nivolumab and the VEGFR inhibitor is the compound of Formula I.
26. The method, medicament, use or kit of embodiment 25, wherein the MK-3475 is formulated as a liquid medicament which comprises 25 mg/ml MK-3475, 7% (w/v) sucrose, 0.02% (w/v) polysorbate 80 in 10 mM histidine buffer pH 5.5 and the VEGFR inhibitor is formulated as the monohydrochloride salt in a 200 mg (by free base) tablet formulated with Magnesium stearate, microcrystalline cellulose, povidone, and sodium starch glycolate.
27. The method, medicament, use or kit of embodiment 25 or 26, wherein the cancer is renal cell carcinoma.
28. The method, medicament, use or kit of embodiment 27, wherein the human is diagnosed with locally advanced or metastatic RCC that is predominantly clear cell histology.
29. The method, medicament, use of kit of embodiment 26 or 27, wherein the human is treatment naïve for one or both of the PD-1 antagonist and the VEGFR inhibitor.
30. The method, medicament, use of kit of any of embodiments 26 to 29, wherein the human does not have a pre-treatment diagnosis or history of any of the following conditions: an autoimmune disease that requires treatment with systemic steroid or immunosuppressive agents, interstitial lung disease, pneumonitis, Malabsorption syndrome, active peptic ulcer disease, inflammatory bowel disease, ulcerative colitis, any gastrointestinal (GI) condition with increased risk of perforation; abdominal fistula, GI perforation, or intra-abdominal abscess.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

### Example 1

### Anti-Tumor Response of Concurrent Administration of a PD-1 Antagonist and Pazopanib to Tumor-Bearing Mice

The anti-tumor therapeutic response of tumor-bearing mice to treatment with one of three regimens: monotherapy with a murine anti-mouse PD-1 monoclonal antibody (Anti-PD1), monotherapy with pazopanib and combination therapy with these two agents administered concurrently.

A cohort of 8 weeks old female BALB/c mice were implanted with 1×10⁶ RENCA cells. When the tumors were ˜115 cubic mm, the tumor-bearing mice were randomized to the following 9 treatment groups of 12 mice per group: (1) 5 mg/Kg Isotype+Vehicle control group; (2) 1 mg/Kg Anti-PD1+Vehicle control; (3) 5 mg/Kg Anti-PD1+Vehicle control; (4) once daily pazopanib+5 mg/Kg Isotype control; (5) once daily pazopanib+5 mg/Kg Anti-PD1; (6) once daily pazopanib+1 mg/Kg Isotype control; (7) once daily pazopanib+1 mg/Kg Anti-PD1; (8) twice daily pazopanib+5 mg/Kg Isotype and (9) twice daily pazopanib+5 mg/Kg Anti-PD1. The Vehicle control was 0.5% hydroxypropylemethylcellulose, 0.1% Tween-80 in distilled water, pH adjusted to 1.3-1.5 with 0.1N hydrochloric acid. The Isotype control was mouse IgG1 specific for an adenoviral hexon protein (clone TC31.27F11). Anti-PD1 was administered to treatment groups 2 and 7 at 1 mg/kg i.p., or groups 3, 5, and 9 at 5 mg/Kg i.p. every 5 days for each of 5 cycles. Pazopanib was administered to treatment groups 4, 5, 6, and 7 at 100 mg/kg once (qd) every day or to treatment groups 8 and 9 at 100 mg/Kg twice (bid) every day for each of 20 cycles.

A one-way ANOVA test was performed to compare tumor volume at the end of study among groups. As demonstrated by the results, which are shown in Figure ______ and Table 4, the mean anti-tumor response of combination therapy with the 5 mg/Kg PD-1 antagonist and twice daily pazopanib (81% tumor growth inhibition in group 9) was greater (p<0.05) than the anti-tumor response observed with either agent used as monotherapy (34% and 52% tumor growth inhibition with groups 2 and 8 respectively).

TABLE 4

| Treatment | Tumor Size | T/C | TGI | |
|---|---|---|---|---|
| (mg/kg) | (D17) mm³)^{a} | (%) | (%) | P value |
| G1: Isotype (5 mpk) + Vehicle | 2,466 ± 154 | — | — | — |
| G2: muDX400 (1 mpk) + Vehicle | 1,675 ± 197 | 66 | 34 | 0.130 |
| G3: muDX400 (5 mpk) + Vehicle | 1,425 ± 243 | 56 | 44 | 0.055 |
| G4: Isotype (5 mpk) + Pazopanib (100 mpk, qd) | 1,555 ± 162 | 61 | 39 | 0.017 |
| G5: muDX400 (5 mpk) + Pazopanib (100 mpk, qd) | 1,052 ± 190 | 40 | 60 | <0.001 |
| G6: Isotype (1 mpk) + Pazopanib (100 mpk, qd) | 1,751 ± 214 | 70 | 30 | 0.309 |
| G7: muDX400 (1 mpk) + Pazopanib (100 mpk, qd) | 1,160 ± 176 | 44 | 56 | <0.001 |
| G8: Isotype (5 mpk) + Pazopanib (100 mpk, bid) | 1,240 ± 97 | 48 | 52 | <0.001 |
| G9: muDX400 (5 mpk) + Pazopanib (100 mpk, bid) | 570 ± 87 | 19 | 81 | <0.001 |

Note:
^{a}Mean ± SEM: P values: G2 vs. G3 = 1.000; G2 vs. G5 = 0.584: G2 vs. G7 = 0.807; G3 vs. G5 = 0.999; G3 vs. G9 = 0.128; G4 vs. G5 = 0.771; G4 vs. G6 = 1.000; G4 vs. G8 = 0.936; G5 vs. G7 = 1.000; G5 vs. G9 = 0.572; G6 vs. G7 = 0.686; G6 vs. G8 = 0.656; G8 vs. G9 = 0.001.

### Example 2

### Clinical Efficacy and Safety of PD-1 Antagonist and VEGFR Inhibitor

A Phase I/II Study is performed to Assess the Safety and Efficacy of Pazopanib and MK-3475 in Subjects with Advanced Renal Cell Carcinoma.

This is an open-label, 2-part study of pazopanib and/or MK-3475 in treatment naïve subjects with advanced RCC. Part 1 consists of a Phase I dose escalation of pazopanib+MK-3475 followed by an expansion cohort to determine the maximum tolerated regimen and the recommended Phase II dose. Part 2 is a randomized 3-arm Phase II study to evaluate the clinical efficacy and safety of pazopanib+MK-3475 as compared to single-agent pazopanib and single-agent MK-3475. The objectives of this Phase I/II study are to test the safety and tolerability of pazopanib in combination with MK-3475, and study the clinical efficacy of pazopanib in combination with MK-3475 in subjects with advanced RCC as compared with single-agent pazopanib and single-agent MK-3475.

| | | | |
|---|---|---|---|
| Part 1: To determine the dose | MTR is defined as the | 8 weeks | No |
| limiting toxicity (DLT) and | highest dose of pazopanib in | | |
| maximum tolerated regimen | combination with the | | |
| (MTR) | highest dose of MK-3475 at | | |
| | which no more than 1 of 6 | | |
| | subjects experiences a DLT | | |
| | after a minimum of 8 weeks | | |
| | of treatment. DLT is defined | | |
| | as a drug-related AE | | |
| | starting in the first 8 weeks | | |
| | of treatment | | |
| Part 1: Number of subjects | | 24 months | No |
| with permanent | | | |
| discontinuation of treatment, | | | |
| dose reductions, interruptions, | | | |
| or delays | | | |
| Part 1: Change from baseline | Laboratory assessments include | Average of | No |
| in laboratory parameters | haematology, clinical chemistry, | 4 years | |
| | urine, coagulation and thyroid | | |
| | function test | | |
| Part 1: Change from baseline | Vital sign measurements will | 30 days | No |
| in vital signs | include heart rate, temperature | after the | |
| | and blood pressure | last dose of | |
| | | study | |
| | | treatment | |
| Part 1: Change from baseline | Cardiac assessments will include | 24 months | No |
| in cardiac parameters | Electrocardiogram (ECG) and | | |
| | Echocardiograms (ECHOs) | | |
| Part 1: Incidence and titer of | Subjects will be monitored for | 24 months | No |
| anti-MK-3475 antibodies | anti-MK-3475 antibodies | | |
| | throughout the study | | |
| Part 2: Progression-free | PFS is defined as the interval | Average of | No |
| survival (PFS) | between the date of randomization | 4 years | |
| | and the earlier date of disease | | |
| | progression (using RECIST v1.1) | | |
| | or death due to any cause. | | |

Secondary Outcomes

| | | | Safety |
|---|---|---|---|
| Measure | Description | Timeframe | Issue |
| Part 1: Dose-escalation | For analysis of plasma | For | No |
| cohorts: pazopanib plasma | pazopanib concentrations, | Pazopanib: | |
| concentrations and serum | blood samples will be collected | before and | |
| MK-3475 concentrations. | and for analysis of serum | after the 1^{st} | |
| | MK-3475 concentrations, | and 2^{nd} dose | |
| | blood samples will be collected | of MK- | |
| | from all subjects | 3475. For | |
| | | MK-3475: | |
| | | Until 6 | |
| | | months after | |
| | | the last dose | |
| | | of MK-3475 | |
| Part 1: Pharmacokinetic (PK) | Area under the plasma | For | No |
| parameters in Expansion | concentration-time curve from | Pazopanib: | |
| cohort | time 0 to 24 hrs (AUC[0-24], | before and | |
| | maximum observed | after the 1^{st} | |
| | concentration (Cmax), tmax, | and 2^{nd} dose | |
| | and concentration at 24 hours | of MK- | |
| | (C24) of pazopanib; pre-dose | 3475. For | |
| | (trough) concentration at the | MK-3475: | |
| | end of the dosing interval | Until 6 | |
| | (Ctau), and apparent clearance | months after | |
| | following oral dosing (CL/F) of | the last dose | |
| | pazopanib will be determined if | of MK-3475 | |
| | data permit. | | |
| Part 1 and Part 2: Overall | Overall response rate is defined | Average of | No |
| response rate (ORR) | as the percentage of subjects, | 4 years | |
| | who achieved either a | | |
| | confirmed complete response | | |
| | (CR) or partial response (PR) | | |
| | by RECIST v1.1 and modified | | |
| | RECIST | | |
| Part 1 and Part 2: Clinical | Clinical benefit rate is defined | Average of | No |
| benefit rate | as a confirmed response of CR | 4 years | |
| | or PR or at least 6-months | | |
| | stable disease by RECIST v1.1 | | |
| | and modified RECIST. | | |
| Part 1 and Part 2: Time to | Time to response is defined for | Average of | No |
| response | all subjects with a confirmed | 4 years | |
| | CR or PR as per | | |
| | RECIST v1.1as the time from | | |
| | randomization until the first | | |
| | documented evidence of CR or | | |
| | PR (whichever status is | | |
| | recorded first) | | |
| Part 1 and Part 2: Duration of | Duration of response is defined | Average of | No |
| response | for all subjects with confirmed | 4 years | |
| | CR or PR as the time from the | | |
| | first documented evidence of | | |
| | CR or PR until time of first | | |
| | documented disease | | |
| | progression or death due to any | | |
| | causes, whichever is first by | | |
| | RECIST v1.1 and modified | | |
| | RECIST | | |
| Part 2: PFS by modified | | Average of | No |
| RECIST | | 4 years | |
| Part 1 and Part 2: Progression- | PFSR₁₈ will be calculated based | 18 months | No |
| free survival rate at 18 months | on Kaplan-Meier estimates of | | |
| (PFSR₁₈) | Progression-free survival (PFS) | | |
| | at 18 months by RECIST v1.1 | | |
| | and modified RECIST | | |
| Part 2: Overall survival (OS) | Overall survival at 18 months | 18 months | No |
| at 18 months | will be summarized based on | | |
| | the Kaplan-Meier method. | | |
| Part 2: Overall survival (OS) | Overall survival will be | Average of | No |
| | summarized using Kaplan- | 4 years | |
| | Meier survival curves | | |
| Part 2: Incidence and severity | | From the | No |
| of adverse events (AEs) and | | start of | |
| serious adverse events (SAEs) | | study | |
| | | treatment | |
| | | (first dose) | |
| | | and, until | |
| | | the post- | |
| | | treatment | |
| | | follow-up | |
| | | visit (at | |
| | | least 30 | |
| | | days after | |
| | | the last dose | |
| | | of | |
| | | investigational | |
| | | product) | |
| | | for AEs, and | |
| | | until 90 | |
| | | days after | |
| | | last dose for | |
| | | SAEs | |
| Part 2: Number of subjects | | Average of | No |
| with permanent | | 4 years | |
| discontinuation of treatment, | | | |
| dose reductions, interruptions, | | | |
| or delays | | | |
| Part 2: Change from baseline | Laboratory assessments include | Average of 4 | No |
| in laboratory parameters | haematology, clinical | years | |
| | chemistry, urine, coagulation | | |
| | and thyroid function test | | |
| Part 2: Change from baseline | Vital sign measurements will | Average of 4 | No |
| in vital signs | include heart rate, temperature | years | |
| | and blood pressure | | |
| Part 2: Change from baseline | Cardiac assessments will | Average of 4 | No |
| in cardiac parameters | include ECG and ECHOs | years | |
| Part 2: Incidence and titer of | Subjects will be monitored for | Until 6 | No |
| anti-MK-3475 antibodies in | anti-MK-3475 antibodies | months after | |
| patients treated with pazopanib + | throughout the study | the last dose | |
| MK-3475 and single-agent | | of MK-3475 | |
| MK-3475 | | | |
| Part 2: PK parameters in | For analysis of plasma | For | No |
| randomized phase | pazopanib concentrations, | Pazopanib: | |
| | blood samples will be collected | Until Dose | |
| | and for analysis of serum | 49 of MK- | |
| | MK-3475 concentrations, | 3475. | |
| | blood samples will be collected | For MK- | |
| | from all subjects. AUC[0-24], | 3475:Until 6 | |
| | Cmax, tmax, and C24 of | months after | |
| | pazopanib alone and in | the last dose | |
| | combination with MK-3475; | of MK-3475 | |
| | Cmax, Ctau, and apparent | | |
| | clearance following oral dosing | | |
| | CL/F of pazopanib will be | | |
| | determined if data permit. | | |

```
Inclusion Criteria:
  Signed written informed consent before performance of study-specific procedures or
assessments and must be willing to comply with treatment and follow-up.
  Diagnosis of locally advanced (defined as disease not amenable to curative surgery or
radiation therapy) or metastatic RCC (equivalent to Stage IV RCC according to American
Joint Committee on Cancer [AJCC] staging) that is predominantly clear cell histology. A
biopsy containing RCC obtained at anytime from the initial diagnosis to study entry
including a recent archival tumor specimen if it is not feasible to obtain a fresh biopsy (a
formalin-fixed, paraffin-embedded [FFPE] tumor block is preferred; tissue from a metastatic
site is acceptable). The tumor tissue must be submitted no later than 10 days before the start
of study treatment.
  Must have measurable disease, i.e. presenting with at least one measurable lesion.
  Subject has received no prior systemic therapy.
  Male or female >=18 years of age or legal age of consent if greater than 18 years.
  A woman is eligible to participate in the study if she is of: Non-childbearing potential
(i.e., physiologically incapable of becoming pregnant), including any female who: Has had a
hysterectomy; Has had a bilateral oophorectomy (ovariectomy), Has had a bilateral tubal
ligation, Is post-menopausal (total cessation of menses for >=1 year); Childbearing potential,
has a negative serum beta-human chorionic gonadotropin (beta-HCG) pregnancy test within
7 days of the first dose of study treatment, not lactating, and agrees to use adequate
contraception during the study until at least 120 days after the last dose of investigational
product..
  Eastern Cooperative Oncology Group performance status (ECOG PS) 0 or 1.
  Adequate organ function as defined in the protocol.
  Left ventricular ejection fraction (LVEF) >= lower limit of normal (LLN) as assessed by
echocardiogram (ECHO) or multigated acquisition (MUGA) scan. The same modality used at
baseline must be applied for subsequent evaluations.
  French subjects: In France, a subject will be eligible for inclusion in this study only if
either affiliated to or a beneficiary of a social security category.
Exclusion Criteria:
  Subject has an active autoimmune disease or a documented history of autoimmune disease
or syndrome that requires systemic steroids or immunosuppressive agents.
  Subject is currently participating or has participated in a study of an investigational agent
or using an investigational device within 30 days of the first dose of study treatment.
  Subject is expected to require any other form of systemic or localized antineoplastic
therapy while on study.
  Subject is on any systemic steroid therapy, within one week before the planned date for
first dose of study treatment. Subject is on any other form of immunosuppressive
medication.
  Subject has a history of a malignancy (other than the disease under treatment in the study)
within 5 years before first study treatment administration..
  Central nervous system metastasis..
  Unable to swallow and retain orally administered medication. Malabsorption syndrome or
disease that significantly affects GI function, or major resection of the stomach or small
bowel that could affect the absorption of pazopanib.
  Subject has interstitial lung disease or a history of pneumonitis.
  Active peptic ulcer disease, inflammatory bowel disease, ulcerative colitis, or other
Gastrointestinal (GI) conditions with increased risk of perforation; history of abdominal
fistula, GI perforation, or intra-abdominal abscess within 4 weeks before beginning study
treatment.
  Known history of human immunodeficiency virus (HIV) infection or a known history of
or is positive for Hepatitis B (Hepatitis B surface antigen [HBsAg] reactive) or Hepatitis C
(HCV Ribonucleic acid [RNA] [qualitative] is detected).
  Presence of active infection requiring systemic therapy.
  Corrected QT interval duration (QTc) prolongation defined as QTc interval >480
milliseconds (msecs).
  History of any one or more of the following cardiac conditions within the past 6 months:
Cardiac angioplasty or stenting; Myocardial infarction; Unstable angina; History of Class III
or IV congestive heart failure according to New York Heart Association (NYHA)
classification.
  History of cerebrovascular accident within the past 6 months.
  Poorly controlled hypertension.
  History of untreated deep venous thrombosis (DVT) (e.g. a calf vein thrombosis that is
not treated or pulmonary embolism within the past 6 months). Note: Subjects with recent
DVT who are treated with therapeutic anti-coagulating agents (excluding therapeutic
warfarin) for at least 2 weeks are eligible.
  Presence of any non-healing wound, fracture, or ulcer, or presence of symptomatic
peripheral vascular disease.
  Evidence of bleeding diathesis or coagulopathy.
  Recent hemoptysis (within 8 weeks before first dose of study treatment).
  Known endobronchial lesions and/or lesions infiltrating major pulmonary vessels that
increase the risk of pulmonary hemorrhage, tumor touching but not infiltrating (abutting) the
vessels is acceptable (CT with contrast is strongly recommended to evaluate such lesions).
  Any serious and/or unstable pre-existing medical, psychiatric, or other conditions that
could interfere with subject's safety, obtaining informed consent or compliance to the study
procedures.
  Previous severe hypersensitivity reaction to another Monoclonal antibody (mAb). Known
immediate or delayed hypersensitivity reaction or idiosyncrasy to drugs chemically related to
the excipients in pazopanib tablets.
  Has taken any prohibited medications that are listed in the protocol within 14 days of the
first dose of study treatment. Subject has received or will receive a live vaccine within 30
days before the first administration of study treatment.
```

Both

| | |
|---|---|
| Minimum age | 18 years |
| Maximum age | NA |
| Healthy Volunteers | No |

Arms & Intervention
Arms

| | | |
|---|---|---|
| Part 1 | Experimental | Part 1 is a dose escalation phase in which subjects |
| | | will receive pazopanib orally and the MK-3475 |
| | | intravenously. Subjects will be evaluated for a |
| | | minimum of 8 weeks before the next dose level |
| | | cohort is enrolled. |
| Part 2 | Experimental | Part 2 is a randomized phase in which subjects |
| | | will be enrolled in each treatment arm: |
| | | Pazopanib monotherapy |
| | | Pazopanib + MK-3475 |
| | | MK-3475 monotherapy |

Intervention

| | | |
|---|---|---|
| Pazopanib | Drug | Pazopanib is an orally administered 200 mg tablet available in |
| | | the dose range of 400 to 800 mg |
| MK-3475 | Drug | MK-3475 is an intravenously administered 100 mg/ |
| | | 4 mL solution available in the potential dose range of |
| | | 1 to 10 mg/kg. |

In Part I of the study, 6 patients have been enrolled in the first cohort with the starting doses of pazopanib 800 mg QD and MK-3475 2 mg/kg Q2W, 4 patients have been enrolled in the second cohort with the starting doses of pazopanib 800 mg QD and MK-3475 2 mg/kg Q3W, 8 patients have been enrolled in the third cohort with the starting doses of pazopanib 600 mg QD and MK-3475 2 mg/kg Q3W and 2 additional patients will be enrolled in this third cohort. So far, preliminary clinical efficacy data (clinical response assessment is based on RECIST 1.1 criteria, starting week 13 following the first dose) are available for the 5 patients in the first cohort, 1/5 confirmed CR, 3/5 PRs (1 confirmed, 2 unconfirmed), 1/5 SD≧6 weeks. One patient in the first cohort was not eligible for clinical efficacy assessment; the patient was withdrew from the study due to DLT at week 9.

| SEQ ID NO: | Description |
|---|---|
| 1 | hPD-1.08A light chain CDR1 |
| 2 | hPD-1.08A light chain CDR2 |
| 3 | hPD-1-08A light chain CDR3 |
| 4 | hPD-1.08A heavy chain CDR1 |
| 5 | hPD-1.08A heavy chain CDR2 |
| 6 | hPD-1.08A heavy chain CDR3 |
| 7 | hPD-1.09A light chain CDR1 |
| 8 | hPD-1.09A light chain CDR2 |
| 9 | hPD-1.09A light chain CDR3 |
| 10 | hPD-1.09A heavy chain CDR1 |
| 11 | hPD-1.09A heavy chain CDR2 |
| 12 | hPD-1.09A heavy chain CDR3 |
| 13 | 109A-H heavy chain variable region |
| 14 | 409A-H heavy chain full length |
| 15 | K09A-L-11 light chain variable region |
| 16 | K09A-L-16 light chain variable region |
| 17 | K09A-L-17 light chain variable region |
| 18 | K09A-L-11 light chain full length |
| 19 | K09A-L-16 light chain full length |
| 20 | K09A-L-17 light chain full length |
| 21 | MK-3475 Heavy chain |
| 22 | MK-3475 Light chain |
| 23 | Nivolumab Heavy chain |
| 24 | Nivolumab light chain |
| 25 | Human PD-L1 |

### REFERENCES

1. Sharpe, A. H, Wherry, E. J., Ahmed R., and Freeman G. J. The function of programmed cell death 1 and its ligands in regulating autoimmunity and infection. Nature Immunology (2007); 8:239-245.
2. Dong H et al. Tumor-associated B7-H1 promotes T-cell apoptosis: a potential mechanism of immune evasion. Nat Med. 2002 August; 8(8):793-800.
3. Yang et al. PD-1 interaction contributes to the functional suppression of T-cell responses to human uveal melanoma cells in vitro. Invest Ophthalmol Vis Sci. 2008 June; 49(6 (2008): 49:2518-2525.
4. Ghebeh et al. The B7-H1 (PD-L1) T lymphocyte-inhibitory molecule is expressed in breast cancer patients with infiltrating ductal carcinoma: correlation with important high-risk propgnostic factors. Neoplasia (2006) 8: 190-198.
5. Hamanishi J et al. Programmed cell death 1 ligand 1 and tumor-infiltrating CD8+ T lymphocytes are prognostic factors of human ovarian cancer. Proceeding of the National Academy of Sciences (2007): 104: 3360-3365.
6. Thompson R H et al. Significance of B7-H1 overexpression in kidney cancer. Clinical genitourin Cancer (2006): 5: 206-211.
7. Nomi, T. Sho, M., Akahori, T., et al. Clinical significance and therapeutic potential of the programmed death-1 ligand/programmed death-1 pathway in human pancreatic cancer. Clinical Cancer Research (2007); 13:2151-2157.
8. Ohigashi Y et al. Clinical significance of programmed death-1 ligand-1 and programmed death-1 ligand 2 expression in human esophageal cancer. Clin. Cancer Research (2005): 11: 2947-2953.
9. Inman et al. PD-L1 (B7-H1) expression by urothelial carcinoma of the bladder and BCG-induced granulomata: associations with localized stage progression. Cancer (2007): 109: 1499-1505.
10. Shimauchi T et al. Augmented expression of programmed death-1 in both neoplasmatic and nonneoplastic CD4+ T-cells in adult T-cell Leukemia/Lymphoma. Int. J. Cancer (2007): 121:2585-2590.
11. Gao et al. Overexpression of PD-L1 significantly associates with tumor aggressiveness and postoperative recurrence in human hepatocellular carcinoma. Clinical Cancer Research (2009) 15: 971-979.
12. Nakanishi J. Overexpression of B7-H1 (PD-L1) significantly associates with tumor grade and postoperative prognosis in human urothelial cancers. Cancer Immunol Immunother. (2007) 56: 1173-1182.
13. Hino et al. Tumor cell expression of programmed cell death-1 is a prognostic factor for malignant melanoma. Cancer (2010): 00: 1-9.
14. Ghebeh H. Foxp3+ tregs and B7-H1+/PD-1+ T lymphocytes co-infiltrate the tumor tissues of high-risk breast cancer patients: implication for immunotherapy. BMC Cancer. 2008 Feb. 23; 8:57.
15. Ahmadzadeh M et al. Tumor antigen-specific CD8 T cells infiltrating the tumor express high levels of PD-1 and are functionally impaired. Blood (2009) 114: 1537-1544.
16. Thompson R H et al. PD-1 is expressed by tumor infiltrating cells and is associated with poor outcome for patients with renal carcinoma. Clinical Cancer Research (2007) 15: 1757-1761.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. §1.57(b)(1), to relate to each and every individual publication, database entry (e.g. Genbank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. A method for treating a cancer in an individual comprising administering to the individual an antagonist of a Programmed Death 1 protein (PD-1) and a VEGFR inhibitor, wherein the VEGFR inhibitor is the compound of Formula I 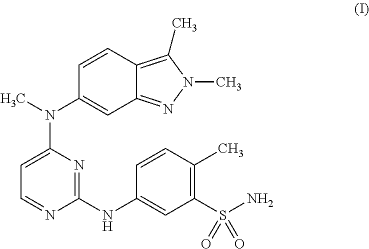 [SMILES: Cc1ccc(Nc2nccc(N(C)c3ccc4c(C)n(C)nc4c3)n2)cc1S(N)(=O)=O]
or a pharmaceutically acceptable salt of the compound of Formula I.

2. The method of claim 1, wherein the individual is a human and the PD-1 antagonist is
(a) a monoclonal antibody, or an antigen binding fragment thereof, which specifically binds to human PD-1 and blocks the binding of human PD-L1 to human PD-1; or
(b) a monoclonal antibody, or an antigen binding fragment thereof, which specifically binds to human PD-L1 and blocks the binding of human PD-L1 to human PD-1.

3. The method of claim 2, wherein the PD-1 antagonist is an anti-PD-1 monoclonal antibody which comprises a heavy chain and a light chain, wherein the heavy and light chains comprise SEQ ID NO:21 and SEQ ID NO:22, respectively, or SEQ ID NO:23 and SEQ ID NO:24, respectively.

4. The method of claim 1, wherein the cancer is a solid tumor.

5. The method of claim 1, wherein the cancer is renal cell carcinoma.

6. The method of claim 1, wherein the PD-1 antagonist is MK-3475 and the VEGFR inhibitor is the HCl salt of the compound of Formula 1.

7. A combination therapy comprising an antagonist of a Programmed Death 1 protein (PD-1) and a VEGFR inhibitor, wherein the VEGFR inhibitor is the compound of Formula I 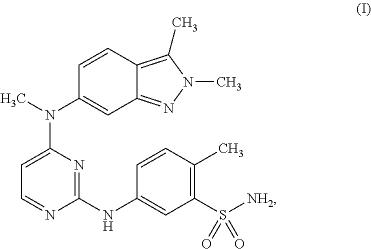 [SMILES: Cc1ccc(Nc2nccc(N(C)c3ccc4c(C)n(C)nc4c3)n2)cc1S(N)(=O)=O]
or a pharmaceutically acceptable salt of the compound of Formula I.

8. (canceled)

9. The combination therapy of claim 7, wherein the individual is a human and the PD-1 antagonist is
(a) a monoclonal antibody, or an antigen binding fragment thereof, which specifically binds to human PD-1 and blocks the binding of human PD-L1 to human PD-1; or
(b) a monoclonal antibody, or an antigen binding fragment thereof, which specifically binds to human PD-L1 and blocks the binding of human PD-L1 to human PD-1.

10. The combination therapy of claim 7, wherein the PD-1 antagonist is an anti-PD-1 monoclonal antibody which comprises a heavy chain and a light chain, wherein the heavy and light chains comprise SEQ ID NO:21 and SEQ ID NO:22, respectively, or SEQ ID NO:23 and SEQ ID NO:24, respectively.

11. The combination therapy of claim 7, wherein the combination therapy is for the treatment of cancer, and the cancer is a solid tumor.

12. The combination therapy of claim 7, wherein the combination therapy is for the treatment of cancer, and the cancer is renal cell carcinoma.

13. The combination therapy of claim 10, wherein the PD-1 antagonist is MK-3475 or nivolumab.

14. The combination therapy of claim 13, wherein the MK-3475 is formulated as a liquid medicament which comprises 25 mg/ml MK-3475, 7% (w/v) sucrose, 0.02% (w/v) polysorbate 80 in 10 mM histidine buffer pH 5.5.

15. The combination therapy of claim 8, wherein the VEGFR inhibitor is formulated as a tablet comprising 200 mg of the compound of Formula I.

16. A kit which comprises a first container, a second container and a package insert, wherein the first container comprises at least one dose of a medicament comprising an antagonist of a Programmed Death 1 protein (PD-1), the second container comprises at least one dose of a medicament comprising a VEGFR inhibitor, and the package insert comprises instructions for treating an individual for cancer using the medicaments, wherein the VEGFR inhibitor is the compound of Formula I

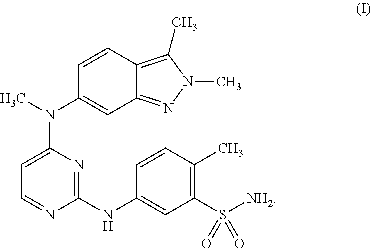 [SMILES: Cc1ccc(Nc2nccc(N(C)c3ccc4c(C)n(C)nc4c3)n2)cc1S(N)(=O)=O]

17. The kit of claim 16, wherein the instructions state that the medicaments are intended for use in treating an individual having a cancer that tests positive for PD-L1 expression by an immunohistochemical (IHC) assay.

18. The kit of claim 16, wherein the individual is a human and the PD-1 antagonist is
(a) a monoclonal antibody, or an antigen binding fragment thereof, which specifically binds to human PD-1 and blocks the binding of human PD-L1 to human PD-1; or
(b) a monoclonal antibody, or an antigen binding fragment thereof, which specifically binds to human PD-L1 and blocks the binding of human PD-L1 to human PD-1.

19. The kit of claim 16, wherein the PD-1 antagonist is MK-3475.

20. The kit of claim 16, wherein the cancer is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

21. The method of claim 1, wherein the compound of formula I is administered once a day, the PD-1 antagonist is administered by IV infusion from every two weeks to every three weeks, and the individual is an adult.

22. The method of claim 1, wherein the compound of formula I is administered twice a day.
